(19) United States
(12) Patent Application Publication
(10) Pub. No.: US 2003/0158132 A1
(43) Pub. Date: Aug. 21, 2003

(54) Method for enhancing bone density or formation

(75) Inventor: Imre Kovesdi, Rockville, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD

(21) Appl. No.: 10/053,637

(22) Filed: Jan. 22, 2002

(51) Int. Cl. A61K 48/00; C12N 5/08

(52) U.S. Cl. 514/44; 424/93.21; 435/366

(57) ABSTRACT

The invention pertains to a method for enhancing bone density or formation. In accordance with the method, a nucleic acid encoding a secreted alkaline phosphatase (SEAP) is administered to a cell in a region of a bone such that the nucleic acid is expressed to produce the SEAP, whereby bone density or formation is enhanced within the region. The method can be employed to produce a bone graft having a cell harboring an exogenous nucleic acid encoding a SEAP. To facilitate the inventive method, the invention provides a recombinant viral vector having a nucleic acid encoding a SEAP. Optionally, a nucleic acid encoding an angiogenic protein and/or a nucleic acid encoding an osteogenic protein is employed in conjunction with the nucleic acid encoding a SEAP.

### TECHNICAL FIELD OF THE INVENTION

This invention pertains to a method and reagents for enhancing bone density or formation.

### BACKGROUND OF THE INVENTION

Most attempts of enhancing bone density or formation have traditionally come in the form of increased support and/or the addition of bone graft material to the site of treatment. Such approaches, however, have had only limited success and often fail to provide aid to patients with bone healing deficiencies. For example, spinal fusion protocols typically employ bone autografts, which are fractured into small pieces and placed between the spinal processes to be fused. Such procedures achieve favorable results only in about 40% of treated patients, and the procedures for harvesting graft material render an already invasive procedure even more so.

Efforts to mimic and/or supplement the normal series of events underlying proper bone healing, and also to cure deficiencies associated with these events, have been forthcoming. For example, blood vessel growth has been stimulated in normally healing rabbit mandibular bones by mixing rabbit bone graft material ex vivo with basic fibroblast growth factor (bFGF) and endothelial cells prior to graft implantation (Eppley et al., J. Oral Maxillofac. Surg., 46, 391-98 (1988)). Moreover, in efforts to accelerate fracture healing, osteoblasts and osseous tissue have been infected in vitro and in vivo with vectors delivering DNA encoding osteogenic proteins, such as transforming growth factor-β1 and bone morphogenic protein-2 (Baltzer et al., Gene Ther., 7, 734-79 (2000); Boden et al., Spine, 23, 2486-92 (1998); Gosdstein et al., Clin. Orthopaed. Rel. Res., 355S, S154-62 (1998); Mehrara et al., J. Bone Min. Res., 14(8), 1290-1300 (1999); Riew et al, Calcif. Tissue Int., 63, 357-60 (1998)). However, many such proteins precipitate an inhibitory effect in treated tissues, and some discourage essential neovascularization within such tissues. Moreover, such protocols requiring treatment of rare cells, such as stem cells, depend on the isolation of sufficient quantities of such cells, which can add yet another level of invasiveness to the procedure, increasing morbidity and post-operative pain and discomfort. Thus, despite improvements in the clinical treatment of bone injuries, there continues to exist a need for improved compositions and/or methods that enhance bone density or formation.

### BRIEF SUMMARY OF THE INVENTION

The invention provides to a method for enhancing bone density or formation. In accordance with the method, a nucleic acid encoding a secreted alkaline phosphatase (SEAP) is administered to a cell in a region of a bone such that the nucleic acid is expressed to produce the SEAP, whereby bone density or formation is enhanced within the region. Optionally, a nucleic acid encoding an angiogenic protein and/or a nucleic acid encoding an osteogenic protein is administered to a cell within the same region such that the nucleic acid is expressed to produce the angiogenic protein and/or the osteogenic protein. The method can be employed to produce a bone graft having a cell harboring an exogenous nucleic acid encoding a SEAP and, optionally, a cell harboring a nucleic acid encoding an angiogenic protein and/or a cell harboring a nucleic acid encoding an osteogenic protein. To facilitate the inventive method, the invention provides a recombinant viral vector having a nucleic acid encoding a SEAP. These and other advantages, as well as additional inventive features, will become apparent after reading the following detailed description.

### DETAILED DESCRIPTION OF THE INVENTION

In accordance with the inventive method, a nucleic acid is administered to a cell (i.e., at least one cell) associated with a desired region of a bone. The relevant “region” of the bone includes the bone itself as well as the immediately adjoining area within the bone or in tissues immediately surrounding it (e.g., periosteum, muscle, fascia, tendons, ligaments, etc.). With this in mind, a cell is “associated with” the bone if it is within the region of the bone before, during, or following application of the inventive method. Any cell associated with the region of the bone can be treated in accordance with the inventive method to express exogenous nucleic acids to produce (and typically secrete) encoded proteins. Inasmuch as such cells are employed as bioreactors in the application of the method, the type of cell is not critical. Thus, the cell generally is any cell type associated with bony structures. Thus, for example, the cell can be within the bone (e.g., a preosteocyte, an osteocyte, chondrocyte, stromal cell, etc.) or in other tissue adjoining the desired region (e.g., a periosteal or fascial cell, a muscle cell, etc.). Thus, the cell can be a cell in vivo. Alternatively, a cell associated with the bone region can be initially away from the region and introduced into it during application of the method. For example, the cell can be within an exogenous tissue (i.e., ex vivo), such as a bone graft or other similar tissue, which is implanted or engrafted into the region of the bone, ultimately in vivo.

The inventive method involves administering a nucleic acid (i.e., first nucleic acid) encoding a secreted alkaline phosphatase (SEAP) to a cell (e.g., a first cell) within the region of the bone. As discussed below, the nucleic acid is delivered to the cell within the region of the bone or prior to its introduction into the region of the bone, such that the nucleic acid is expressed in the cell to produce the secreted protein. The presence of the SEAP, in turn, enhances bone density or formation within the region of the bone.

Nucleic acid sequences encoding a SEAP are known (e.g., SEQ ID NOS: 1-4; see also, e.g., Berger et al., Gene, 66, 1-10 (1988); International Patent Application WO 99/10525), and any of these, as well as secreted active derivatives of an alkaline phosphatase protein (as described in, e.g., Coleman, Annu. Rev. Biophys. Biomol. Struct., 21, 441-83 (1992); Lowe, J. Cell. Biol., 116 (3), 799-807 (1992); Fishman, Clin. Biochem., 23(2), 99-104 (1990); Kishi et al., Nucleic Acids Res., 17(5), 2129 (1989); Harris, Clin. Chem. Acta, 186, 133-150 (1989); Millan, Anticancer Res., 8, 995-1004 (1988); Weiss et al., J. Biol. Chem., 263(24), 12002-12010 (1988); Coleman et al., Adv. Enzymol., 55, 381 (1983); and U.S. Pat. Nos. 4,659,666 and 5,434,067), can suitably be employed in the context of the inventive method. In this regard, for example, a known alkaline phosphatase gene (see, e.g., Henthorn et al., Proc. Natl. Acad. Sci. U.S.A., 83, 5597-5601, (1986); Kam, et al., Proc. Natl. Acad. Sci. U.S.A., 82, 8715-8719 (1985); Knoll et al., Gene, 60, 267-76 (1987); Knoll et al., J. Biol. Chem., 263(24), 12020-12027 (1988); Martin et al., Nuc. Acids Res., 15, 9104 (1987); Millan et al., J. Biol. Chem., 261, 3112-15 (1986); Rump et al., Genomics, 73, 50-55 (2001)) can be modified or engineered to include a functional secretion leader sequence, and/or the approximately 11-25 carboxy-terminal residues can be deleted, which permits passage of the protein through the membrane of producing cells (see, e.g., European Patent Publication 0327960). For example, the alkaline phosphatase peptide portion can be a human alkaline phosphatase, a non-human alkaline phosphatase (as described in, e.g., U.S. Pat. No. 5,980,890), or a biologically active fragment or homolog thereof (e.g., a synthetic alkaline phosphatase). In addition to preferably retaining the 10-strand mixed beta sheet structure associated with mammalian alkaline phosphatases, the alkaline phosphatase peptide portion desirably retains a zinc ion binding domain (typically, the carboxyl end of the central beta sheet) and magnesium ion binding domain of a wild-type alkaline phosphatase or homolog thereof, and preferably exhibits zinc and magnesium ion binding within similar binding coordinates (e.g., differing by less than about 0.5 angstrom, preferably less than about 0.1 angstrom) as a wild-type alkaline phosphatase (as described in, e.g., Coleman, Annu. Rev. Biophys. Biomol. Struct., 21, 441-83 (1992)). The alkaline phosphatase peptide portion desirably forms multimers with alkaline phosphatases or other alkaline phosphatase peptide portion-containing fusion proteins, desirably in which at least one multimer member binds a zinc ion in addition to the alkaline phosphatase peptide portion. Also advantageously, the alkaline phosphatase portion exhibits biological activity similar to a wild-type alkaline phosphatase (e.g., substrate binding as described with respect to select alkaline phosphatases in U.S. Pat. No. 5,783,567 and/or hydrolysis of monophosphate esters, particularly under physiological alkaline conditions (i.e., above a pH of about 7.4)). Preferably, the alkaline phosphatase peptide portion reacts with at least one alkaline phosphatase antibody. Examples of techniques for determining if a bone alkaline phosphatase will react with a bone alkaline phosphatase antibody are provided in U.S. Pat. No. 6,201,109, which can be modified with respect to other alkaline phosphatase peptide portions (e.g., a SEAP) as necessary.

Preferably, the alkaline phosphatase peptide portion exhibits at least about 40% homology (preferably at least about 45% homology, and more preferably at least about 45% identity) to a human alkaline phosphatase (e.g., intestinal alkaline phosphatase, placental alkaline phosphatase, placental-like alkaline phosphatase, liver/bone/kidney alkaline phosphatase (tissue non-specific alkaline phosphatase), and the like), and desirably exhibits at least about 70% weight homology, and more preferably at least about 80% weight homology, to a human wild-type alkaline phosphatase. The alkaline phosphatase may or may not include an alkaline phosphatase signal sequence (such as the human SEAP signal sequence), and may or may not include the alkaline phosphatase propeptide sequence (e.g., the human SEAP propeptide sequence). Desirably, the alkaline phosphate portion comprises a sequence exhibiting at least about 60%, more preferably at least about 70%, identity to residues 65-172 of human SEAP (SEQ ID NO: 2). Desirably, the alkaline phosphatase peptide portion will be about 100-700, more preferably about 200-550, and even more preferably about 500 amino acid residues in length. The alkaline phosphatase may comprise or lack sequences associated with lipid association, glycosylation, or both present in wild-type alkaline phosphatases (e.g., the N₁₄₄-associated glycosylation site and/or D₅₀₆ lipid-binding GPI-anchor site). As the alkaline phosphatase is preferably secreted, it will desirably lack a transmembrane domain (e.g., the SEAP precursor transmembrane domain), or functional equivalent or sequence homolog thereof Alternatively, the alkaline phosphatase can be rendered in secreted form through small residue changes, including even single residue substitutions, as is known in the art. In another embodiment, a gene encoding a typically membrane-bound alkaline phosphatase can be modified by removing that portion of the gene that encodes the transmembrane anchor of the protein and adding a stop codon, thus permitting secretion

To enhance the efficacy of the inventive method, a nucleic acid (i.e., second nucleic acid) encoding an angiogenic protein can be similarly delivered to a cell (e.g., a second cell) within the same region of the bone as is the nucleic acid encoding a SEAP. In this context, an angiogenic protein is any protein that potentiates or enhances neovascularization, many of which are known in the art. While any such factor can be employed in the context of the inventive method, because VEGF proteins are not known to induce the growth of tissues not involved in the production of new vasculature, a preferred angiogenic protein is a VEGF protein (e.g., VEGF-A, VEGF-B, VEGF-C, VEGF-D, VEGF-E, VEGF2), and more preferably VEGF₁₂₁, VEGF₁₃₈, VEGF₁₄₅, VEGF₁₆₂, VEGF₁₆₅, VEGF₁₈₂, VEGF₁₈₉, or an derivative or fragment thereof (see, e.g., U.S. Pat. Nos. 5,332,671 (Ferrara et al.), 5,240,848 (Keck et al.); and 5,219,739 (Tischer et al.)). Most preferably, because of their higher biological activity, the angiogenic protein is VEGF₁₂₁ or VEGF₁₆₅, particularly VEGF₁₂₁. Inasmuch as VEGF₁₂₁ typically binds heparin with lesser affinity than does VEGF₁₆₅, VEGF₁₂₁ is particularly preferred for use in the inventive method. While VEGF proteins are preferable for use in the inventive method, other angiogenic proteins include connective tissue growth factor (CTGF), fibroblast growth factors (FGFs) (e.g., aFGF, bFGF, and FGF-1-23), angiopoiteins, angiopoetin homologous proteins, angiogenin, angiogenin-2, and P1GF (see, e.g., U.S. Pat. Nos. 5,194,596, 5,219,739, 5,338,840, 5,532,343, 5,169,764, 5,650,490, 5,643,755, 5,879,672, 5,851,797, 5,843,775, and 5,821,124; International Patent Application WO 95/24473; European Patent Documents 476 983, 506 477, and 550 296; Japanese Patent Documents 1038100, 2117698, 2279698, and 3178996; and J. Folkman et al., Nature, 329, 671 (1987)).

A nucleic acid (i.e., third nucleic acid) encoding an osteogenic protein can be similarly delivered to a cell (e.g., a third cell) within the same region of the bone as is the nucleic acid encoding a SEAP and optionally the nucleic acid encoding the angiogenic factor. In this context, an osteogenic protein is any protein that potentiates or enhances ossification or differentiation of bone, many of which are known in the art. Osteogenic proteins include, for example, systemic hormones (e.g., parathyroid hormone (PTH) estrogen, etc.), growth factors (e.g., CTGF and CTGF-like growth factor), cytokines, chemotactic and adhesive proteins, molecules such as activin (U.S. Pat. No. 5,208,219), bone morphogenic proteins (BMPs), growth factor receptors, and the like. Preferably, the osteogenic protein is a bone morphogenic protein (BMP) (e.g., BMP-2, BMP-3, BMP-4, BMP-5, BMP-6, BMP-7 BMP-8, and BMP-9), a transforming growth factor (TGF) (e.g., TGF-β1), a latent TGF binding protein (LTBP), latent membrane protein-1 (LMP-1), a heparin-binding neurotrophic factor (HBNF), growth and differentiation factor-5 (GDF-5), a parathyroid hormone (PTH), a fibroblast growth factor (FGF), an epidermal growth factor (EGF), a platelet-derived growth factor (PDGF), an insulin-like growth factor (e.g., IGF-1 or 2)), a growth factor receptor, a cytokine, a chemotactic factor, a granulocyte/macrophage colony stimulating factor (GMCSF), a LIM mineralization protein (LMP) (Boden et al., Spine, 23, 2486-92 (1998)), a leukemia inhibitory factor (LIF), a hedgehog protein (e.g., Desert Hedgehog (DHH), Indian Hedgehog (IHH), Sonic Hedgehog (SHH), etc.), or an active derivative or fragment thereof. Most preferably, the osteogenic protein is TGF-β1 or midkine (MK), and these are preferably employed where the angiogenic factor is a VEGF. Some osteogenic proteins also can stimulate the growth or regeneration of skeletal connective tissues such as, e.g., tendon, cartilage, ligament, etc.

Where a nucleic acid encoding an angiogenic protein and/or a nucleic acid encoding an osteogenic protein (i.e., second and third nucleic acids) are employed in the inventive method in addition to the nucleic acid encoding a SEAP, the nucleic acids can be delivered to the same or different cells associated with the region of the bone. In this respect the first cell and the second cell can be the same cells or different cells, as can the third cell. The method can be more efficacious if the nucleic acid(s) are delivered to many cells associated with the region of the bone, such as a population of cells, or a majority of cells within the region. In any event, within at least the first cell, the first nucleic acid is expressed, leading to the production of the SEAP. Desirably, a second nucleic acid is employed to similarly lead to the production of the angiogenic protein, and a third nucleic acid is employed to lead to the production of the osteogenic protein. Typically the encoded protein(s) is (are) secreted from the cell, but this is not a requirement. The presence of the SEAP (and desirably the angiogenic protein and/or the osteogenic protein) promotes physiological changes within the region of the bone so as to enhance bone density or formation.

While the sequences of SEAP and many angiogenic and osteogenic proteins, as well as nucleic acids encoding them, are known, any active derivative sequence can be employed in the place of known sequences. These derivatives can be naturally occurring or engineered (e.g., synthetically produced) and include those caused by point mutations, those due to the degeneracy of the genetic code or naturally occurring allelic variants, and further modifications that are introduced by genetic engineering. Indeed, a preferred embodiment of the invention employs a fusion protein comprising a first SEAP domain and a second angiogenic or osteogenic domain. Indeed, such a fusion protein can have three domains: a SEAP domain, an angiogenic domain, and an osteogenic domain. In this respect, the SEAP encoded by the nucleic acid used in the method of the invention can be the same protein as the osteogenic protein and/or the angiogenic protein.

To facilitate the inventive method, the invention provides a recombinant expression cassette encoding a fusion protein having a first SEAP domain and a second angiogenic domain and/or osteogenic domain. Such a fusion protein can be or comprise, for example, a SEAP/MK fusion protein, a SEAP/HBNF fusion protein, a SEAP/VEGF fusion protein, or the fusion of SEAP and any of the factors listed herein. The expression cassette encoding a SEAP fusion protein can be generated by standard methods. For example, the coding sequence of either SEAP (or its desired fusion partner) can be genetically modified to include one or more restriction endonuclease cleavage sites, at least one of which is placed in-frame at a point in the coding sequence or at the stop codon. A nucleic acid fragment encoding the desired portion of the coding sequence of the desired fusion partner (or SEAP) can be engineered to contain ends compatible with the restriction endonuclease cleavage sites (e.g., by PCR using primers having the desired compatible sequence or by first cloning the desired sequence into a polylinker having flanking compatible sequences). This approach facilitates restriction digests of the SEAP and the desired fusion partner, the products of which then can be ligated to form a single coding polynucleotide encoding a polypeptide having a first SEAP domain and a second angiogenic and/or osteogenic domain (i.e., the fusion partner). A preferred fusion partner is decorsin, which is a high affinity antagonist of the avb3 and aIIbb3 receptors (Krezel et al., Science, 264, 1944-47 (1994)). It should be noted that any or all (e.g., either or both) of the domains can be somewhat truncated by this process, but such truncations should not be so extreme as to result in an inoperative domain. It is, thus, to be expected that each domain retains the SEAP, angiogenic, or osteogenic function, which can be assessed by standard methodology (e.g., in vitro testing for phosphatase activity, or in vivo testing for angiogenesis and/or osteogenesis).

Another method of generating the expression cassette encoding a SEAP fusion protein is to generate a population of polynucleotides containing SEAP coding sequences via PCR using defined 3′ and 5′ primers and also to generate a second population of polynucleotides containing sequences encoding the desired fusion partner via PCR using defined 3′ and 5′ primers. The two populations of polynucleotides then can be mixed and employed as a template for another round of PCR using the 5′ primer of one of the populations (i.e., the SEAP or the desired fusion partner) and the 3′ primer of the other. Some fraction of the PCR products will represent fill-in or annealed fusion polynucleotides encoding both SEAP and the desired fusion partner.

Methods other than these two exemplary methods can be employed to generate polynucleotides encoding the SEAP fusion protein. The sequence then can be cloned by standard techniques into any desired vector, which can also possess other genetic elements as set forth herein. Of course, however generated, it is desirable to assess the sequence of the resulting expression cassette, such as by determining its sequence and verifying its expressibility and the function of the encoded fusion protein.

Regardless of the species of reagent (e.g., SEAP, angiogenic protein, osteogenic protein, or fusion of one or more such proteins), successful application of the inventive method enhances bone density or formation in some respect. Thus, it is to be understood that the inventive method can strengthen or harden a region of contiguous bone. In other applications, enhancement of bone density or formation is associated with healing (e.g., fusion) of a splintered or fractured bone. Similarly, the inventive method can facilitate fusion of two bone masses, such as a bone graft to a bony region within a patient or the fusion of separate bones within a patient, such as vertebrae or other desired bony structures. Moreover, in certain embodiments, the inventive method can be employed to stimulate the growth or repair of both bone tissue itself and also of skeletal connective tissues that surround or are associated with bone. Thus, the method can facilitate the attachment of such bone-associated tissues (e.g., ligaments) to bones.

A nucleic acid employed in the inventive method can be any suitable type sufficient to lead to the production of the desired protein within the cell(s) associated with the desired region of bone. In this respect, a nucleic acid can be RNA, cDNA, genomic DNA, etc., but typically it is cDNA, such as, for example, within an expression cassette. Moreover, in embodiments in which a nucleic acid encoding a SEAP is delivered in conjunction with a nucleic acid encoding an angiogenic protein and/or a nucleic acid encoding an osteogenic protein, the two (or three) nucleic acids can be present in the same molecule or on separate molecules (i.e., the first nucleic acid and the second nucleic acid can be the same, the first and third nucleic acids can be the same, and all three nucleic acids can be the same). Of course, inasmuch as these nucleic acids can be delivered to different cells (i.e., first, second, and/or third cells), the two or three coding nucleic acids can be present on separate molecules (e.g., vectors).

Where a nucleic acid for use in the inventive method is within an expression cassette, the cassette also should comprise a promoter able to drive the expression of the coding sequence within the cells. Many viral promoters are appropriate for use in such an expression cassette (e.g., retroviral ITRs, LTRs, immediate early viral promoters (IEp) (such as herpesvirus IEp (e.g., ICP4-IEp and ICP0-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters)). Other suitable promoters are eukaryotic promoters, such as enhancers (e.g., the rabbit β-globin regulatory elements), constitutively active promoters (e.g., the β-actin promoter, etc.), signal specific promoters (e.g., inducible and/or repressible promoters, such as a promoter responsive to Tetracycline or RU486, the metallothionine promoter, etc.), and tissue-specific promoters. Moreover, where the first, second, and/or third nucleic acids are part of the same molecule, their respective cassettes can share a bi-directional promoter, many of which are known in the art (see, e.g., Lee et al., Mol Cells., 10(1), 47-53 (2000); Dong et al., J. Cell. Biochem., 77(1), 50-64 (2000); and Li et al., J. Cell. Biochem., 273(43), 28170-77 (1998)), such that the respective coding sequences can be on opposite strands of the molecule (see, e.g., International Patent Applications WO 99/15686 and WO 98/56937).

Regardless of the type of promoter employed, within the expression cassette, the coding polynucleotide(s) and the promoter(s) are operably linked such that the promoter(s) is(are) able to drive the expression of the desired sequence(s). As long as this operable linkage is maintained, the expression cassette can include more than one gene, such as multiple coding sequences (e.g., the first, second, and/or third nucleic acids, as discussed herein) separated by ribosome entry sites. Furthermore, the expression cassette can optionally include other elements, such as polyadenylation sequences, transcriptional regulatory elements (e.g., enhancers, silencers, etc.), or other sequences.

For successful application of the inventive method, a nucleic acid encoding the SEAP and optionally the nucleic acid(s) encoding the angiogenic protein and/or the osteogenic protein must be introduced into a cell associated with the desired region of the bone in a manner suitable for it to be expressed and to produce the encoded sequence. Any suitable vector can be employed to this end, many of which are known in the art. Examples of such vectors include naked RNA and DNA vectors (such as oligonucleotides, artificial chromosomes (e.g., yeast artificial chromosomes (YACs)), cosmids, plasmids, etc.), viral vectors such as adeno-associated viral vectors (Berns et al., Ann. N.Y. Acad. Sci., 772, 95-104 (1995)), adenoviral vectors (Bain et al., Gene Therapy, 1, S68 (1994)), herpesvirus vectors (Fink et al., Ann. Rev. Neurosci., 19, 265-87 (1996), U.S. Pat. Nos. 5,837,532, 5,846,782, 5,849,572, and 5,804,413, and International Patent Applications WO 91/02788, WO 96/04394, WO 98/15637, and WO 99/06583), packaged amplicons (Federoff et al., Proc. Nat. Acad. Sci. USA, 89, 1636-40 (1992)), papilloma virus vectors, phage vectors, picornavirus vectors, polyoma virus vectors, retroviral vectors, SV40 viral vectors, vaccinia virus vectors, and other vectors. While some of the indicated vectors are suitable for use only with certain types of polynucleotides (e.g., cDNA as opposed, for example, to RNA), the selection of an appropriate vector and the use thereof to introduce exogenous genetic material (e.g., the desired nucleic acids) into cells are within the skill of the art.

Once a given type of vector is selected, its genome must be manipulated for use as a background vector, after which it must be engineered to incorporate exogenous polynucleotides. Methods for manipulating the genomes of vectors are well known in the art (see, e.g., Sambrook et al., Molecular Cloning: A Laboratory Manual, 2d edition, Cold Spring Harbor Press (1989); Ausubel et al., Current Protocols in Molecular Biology, Greene Publishing Associates and John Wiley & Sons, New York, N.Y. (1994)) and include direct cloning, site specific recombination using recombinases, homologous recombination, and other suitable methods of constructing a recombinant vector. In this manner, an expression cassette can be inserted into any desirable position of the vector. Moreover, in addition to the desired expression cassette, a vector also can include other genetic elements as appropriate, such as, for example, genes encoding a selectable marker (e.g., β-gal or a marker conferring resistance to a toxin, such as puromycin or other similar selectable markers), a pharmacologically active protein, a transcription factor, or other biologically active substance.

As mentioned, within the context of the inventive method, the nucleic acid encoding the SEAP and optionally the nucleic acid(s) encoding the angiogenic protein and/or the osteogenic protein can be delivered to the cells as (i.e., within) a viral vector. To facilitate such embodiments of the method, the invention provides a viral vector having a nucleic acid (i.e., a first nucleic acid) encoding a SEAP. Inasmuch as the nucleic acid encoding a SEAP can be present in the same molecule as a nucleic acid encoding an angiogenic protein (i.e., second nucleic acid) and/or a nucleic acid encoding an osteogenic protein (i.e., third nucleic acid) so too can the inventive viral vector have a second nucleic acid encoding an angiogenic protein and/or a third nucleic acid encoding an osteogenic protein, in addition to the first nucleic acid encoding a SEAP.

While the inventive viral vector can be any suitable type of virus, adenoviral vectors present several advantages, particularly for in vivo applications, not the least of which is that the knowledge of such vectors has advanced to a stage where virulence can be eliminated, tropism can be altered, exogenous genetic material can be introduced into such viral backbone, and the virus can be efficiently constructed, grown, purified, and stored (see, e.g., U.S. Pat. Nos. 6,063,627, 6,057,155, 6,013,638, 5,997,509, 5,994,106, 5,965,541, 5,965,358, 5,962,311, 5,928,944, 5,869,037, 5,851,806, 5,849,561, 5,846,782, 5,837,511, 5,801,030, 5,770,442, 5,731,190, 5,712,136, and 5,559,099; International Patent Applications WO00/34496, WO00/34444, WO00/23088, WO00/15823, WO00/12765, WO00/00628, WO99/55365, WO99/54441, WO99/41398, WO99/23229, WO99/15686, WO98/56937, WO98/54346, WO98/53087, WO98/40509, WO98/32859, WO98/07877, WO98/07865, WO97/49827, WO97/21826, WO97/20051, WO97/12986, WO97/09439 and WO 96/26281, WO 96/07734, and WO 95/34671; and European Patent Documents 0863987, 0866873, 0870049, 0914459, 0920524, 0973927, 0988390, 0996735, 1012291, and 1015620). Indeed, recombinant adenoviruses having angiogenic genes are known in the art (see, e.g., Mack et al., J. Thorac. Cardiovasc. Surg., 115(1), 168-76 (1998); Magovern et al, Hum. Gene. Ther., 8(2), 215-27 (1997)), and a nucleic acid encoding a SEAP or an osteogenic protein can be cloned into such a backbone vector by standard methods.

Given the state of the art, an adenoviral vector of the invention can be derived from any desired serotype of adenovirus. Adenoviral stocks that can be employed as a source of adenovirus can be amplified from the adenoviral serotypes 1 through 51, which are currently available from the American Type Culture Collection (ATCC, Rockville, Md.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Preferably, however, an adenovirus is of serotype 2, 5 or 9.

Typically, aside from containing the nucleic acid encoding a SEAP (and optionally the second and/or third nucleic acids encoding the angiogenic protein and the osteogenic protein, respectively), the viral vector is deficient in at least one gene function essential (i.e., required) for viral replication. Such a viral vector generally is unable to replicate except in cells engineered to provide (i.e., complement for) the missing essential gene function(s). For example, an adenoviral vector can have at least a partial deletion of the E1 (e.g., E1a or E1b), E2, and/or E4 regions so as to provide a vector deficient in at least on essential gene function in one or more regions. Desirably such a virus has a deletion (particularly a deletion sufficient to impair at least one essential gene function) in two, three, or even all of these regions. Suitable replication-deficient adenoviral vectors are disclosed in U.S. Pat. Nos. 5,851,806 and 5,994,106 and International Patent Applications WO 95/34671 and WO 97/21826. Indeed, in preferred embodiments, at least one of the exogenous nucleic acids (e.g., encoding the angiogenic and/or osteogenic proteins) is cloned into the E1 region of the adenoviral backbone, desirably oriented from “right to left” within the adenoviral genome (which otherwise is oriented “left to right”). While the E3 region is not essential for viral replication, the inventive adenoviral vector also can have at least a partial deletion in the E3 region as well.

In addition to a deficiency in the E1, E2, E3, and/or E4 regions, an adenoviral vector according to the invention also can have a mutation in the major late promoter (MLP), for example in one or more control element(s) such that it alters the responsiveness of the promoter (see, e.g., U.S. Pat. No. 6,113,913). Moreover, the tropism of viral vectors can be altered, for example by incorporating chimeric coat proteins into a viral surface that contain ligands able to mediate viral attachment to cell surfaces (e.g., either directly or through a bi- or multi-specific molecule) and/or by destroying the native tropism of the virus. Where the tropism of the virus is altered from that of the source virus, preferably it is engineered to contain a ligand conferring the ability to bind cells associated with bone tissue, such as, for example, osteocytes, chondrocytes, periosteal cells, myocytes, and cells in muscle and tendons that are associated with the type of bone to be treated. Many such ligands are known, and techniques for generating replication deficient adenoviral vectors and for altering viral tropism are well known in the art. A preferred ligand contains a short stretch of positively charged residues, as such ligands are able to bind integrin molecules present on many cell types (see, e.g., U.S. Pat. No. 5,965,541).

In application, the first and optionally the second and/or third nucleic acids (or a virus containing them, if appropriate) are delivered to the cell within a physiologically acceptable solution. Accordingly, to facilitate the inventive method, the invention provides a pharmaceutical (including pharmacological) composition including a first nucleic acid encoding a SEAP and optionally a second nucleic acid encoding an angiogenic protein and/or a third nucleic acid encoding an osteogenic protein (any of which, of course, can be within a recombinant virus as described herein), and a diluent. The diluent can include one or more pharmaceutically- (including pharmacologically- and physiologically-) acceptable carriers. For compositions suitable for in vitro application, the diluent can be a suitable tissue culture medium. Pharmaceutical compositions for use in accordance with the invention can be formulated in any conventional manner using one or more pharmaceutically acceptable carriers comprising excipients, as well as optional auxiliaries that facilitate processing of the nucleic acids into preparations which can be used pharmaceutically. Proper formulation is dependent upon the route of administration chosen. Thus, for systemic injection, the nucleic acids can be formulated in aqueous solutions, preferably in physiologically compatible buffers. The nucleic acids can be formulated for parenteral administration by injection, e.g., by bolus injection or continuous infusion. Such compositions can take such forms as suspensions, solutions or emulsions in oily or aqueous vehicles, and can contain formulatory agents such as suspending, stabilizing and/or dispersing agents. For application to bony tissues, a preferred composition includes a porous or spongy matrix, such as collagen, which can be soaked or perfused with a fluid or semifluid carrier (e.g., buffered saline solution) including the nucleic acid(s). Such a matrix material assists in retaining the nucleic acid(s) within the site of the bone to be treated. Of course, the nucleic acid(s) also can be formulated into other compositions appropriate to the vector type such as those known in the art. Thus, for example, the nucleic acid(s) can be administered in combination with further agents, such as, e.g., liposomes, lipids (e.g., cationic or anionic lipids), polypeptides, or various pharmaceutically active agents. In some embodiments, the nucleic acid(s) can be delivered along with various other agents, such as an angiogenic factor and/or an inhibitor of bone resorbtion (see, e.g., U.S. Pat. Nos. 5,270,300 and 5,118,667). A preferred formulation is described in U.S. Pat. No. 6,225,289.

The composition(s) containing the nucleic acid(s) (or viral vector(s)) is(are) delivered to tissue associated with the region of bone to be treated at any dose appropriate to enhance bone density or formation within the region. The appropriate dose will vary according to the type of vector employed, but it is within routine skill to select a suitable dosage. Thus, for example, where the nucleic acids are within an adenoviral vector, a dose typically will be at least about 1×10⁵ pfu (e.g., 1×10⁶-1×10¹² pfu) to the site of administration. The dose preferably is at least about 1×10⁷ pfu (e.g., about 1×10⁷-1×10¹² pfu), more preferably at least about 1×10⁸ pfu (e.g., about 1×10⁸-1×10¹¹ pfu), and most preferably at least about 1×10⁹ pfu (e.g., about 1×10⁹-1×10¹⁰ pfu). For purposes of considering the dose in terms of particle units (pu), also referred to as viral particles, typically from about 10 to about 100 particles is equivalent to about 1 pfu (e.g., 1×10¹² pfu is roughly equivalent to 1×10¹⁴ pu). In a single round of vector administration, using, for example, an adenoviral vector deleted of the E1a region, part of the E1b region, and part of the E3 region of the adenoviral genome, wherein the vector contains a nucleic acid sequence(s) encoding SEAP and optionally human VEGF₁₂₁ or VEGF₁₆₅ under the control of a standard CMV immediate early promoter, about 10⁷-10¹² pfu, preferably about 10⁹-10¹⁰ pfu, are administered to the desired region. Of course, the amount of virus administered can vary depending on the volume of the area to be treated.

The composition(s) is (are) administered to the region of the bone in an appropriate manner to deliver the nucleic acid(s) to the tissue. For application in vivo, the region of the bone can be exposed, and the composition can be delivered, so as to be in physical contact with the tissues within the region. While in some applications it is desirable to expose the bone itself, in other applications tissue surrounding or otherwise associated with the bone can be retained intact, with the composition delivering the nucleic acids to cells existing within such tissues. Inasmuch as the method can be employed in conjunction with standard surgical techniques, its application can be directed to serve any desired treatment goal. For example, the method can be employed to promote fracture repair by delivering the composition to the region of the fracture. Alternatively, the method can be employed with methods for bone fusion (e.g., vertebral fusion).

For application in vitro, such as on a bone graft, the bone material can be bathed in a composition containing the nucleic acid(s) or, where appropriate, the bone material can be perfused with the composition. The period of such bathing or perfusion should be sufficient so as to permit the cell or cells to take up the nucleic acid(s). Depending on the desired use of the graft and the genetic constructs employed (e.g., inducible promoters, etc.), the cells need not be induced to express the nucleic acids in vitro.

Where the method is employed in vitro, it can be used to create bone grafts for tissue repair. Accordingly, the invention provides a bone graft having a first cell (preferably a population of cells) having a first exogenous nucleic acid encoding a SEAP. Optionally, the bone graft can have a second cell (preferably population of cells) having a second exogenous nucleic acid encoding an angiogenic protein and/or a third cell (preferably a population of cells) having a third nucleic acid encoding an osteogenic protein, such as are described above. Within the graft, the first, second, and/or third cells can be the same, and the first, second, and/or third populations of such cells can partially or completely overlap (i.e., contain cells of another population). Similarly, as described above, the first, second, and/or third nucleic acids can be the same.

The graft can be obtained from any suitable donor source according to commonly employed surgical techniques. The iliac crest is a common source of tissue for bone grafts. Alternatively, the graft can be grown de novo (e.g., from osteocytes, preosteocytes, stem cells, cartilage, etc.) prior to treatment. In this respect, the graft can be an autograft, derived from any desirable bony structure in the patient to whom the graft is to be re-implanted. In other applications, the graft can be an allograft or even a xenograft. Indeed, such graft tissue can be preserved for use in future applications, e.g., through incubation in culture medium, refrigeration, cryopreservation, etc. In any event, after the nucleic acids have been transferred to a cell or cells within the graft, it can be implanted into a patient according to standard surgical techniques. Where the graft is other than an autograft, however, appropriate immunosupression should be employed as necessary to mitigate graft rejection. The cell(s) within the graft to which the nucleic acids have been transferred should express the nucleic acids to produce the SEAP and optionally the angiogenic and/or osteogenic proteins at least after the graft has been implanted into the patient. As discussed, the presence of such proteins within the region of the fissure between the graft and the host tissue will facilitate fusion of the graft to the host bone.

### EXAMPLE 1

This example describes the construction of an adenovirus vector containing an expression cassette encoding a SEAP.

The SEAP coding sequence (SEQ ID NO: 3) was cloned from TROPIX™ pBC12/PL/SEAP VECTOR™, which contains the SEAP coding sequence having a mutation incorporating a HpaI site and a stop codon. In cloning the sequence, a 5′ oligonucleotide was employed to mutate the sequence to have a HindIII restriction recognition site, such that the Met 21 of the native sequence (SEQ ID NO: 2) would be the first encoded amino acid. The resulting cloned nucleic acid, and the encoded protein, are set forth at SEQ ID NOS: 5 and 6. The nucleic acid sequence was further modified by changing the 3′ XhoI site to HindIII using a oligonucleotide linker. Thereafter, the SEAP sequence was cloned into a pAdCMV.MCS adenovirus transfer vector, which includes nucleotides 1-5790 of the adenoviral serotype 5 genome, except nucleotides 355-3332 (which encompass the adenoviral E1A and E1B coding regions), the CMV promoter, a multiple cloning site (including a HindIII site), the SV40 poly A site, and a splice donor/acceptor site between Ad5 nucleotides 355 and 3332.

After insertion of the SEAP fragment, the recombinant transfer vector was used to generate a transfection plasmid capable of producing an E1-deleted adenoviral vector containing the SEAP-encoding sequence positioned in the E1-deleted region upon transfection into a suitable host cell. The transfection plasmid was transfected into an E1 complementing cell line, 293 cells, using standard techniques, thereby resulting in the production of a stock of E1-deleted, replication-deficient, adenoviral vectors containing the SEAP-encoding nucleic acide sequence (AdSEAP). Preferably, the vector-cell line system selected is such that replication competent adenovirus (RCA) levels in the stock are less than about 1×10⁷ plaque forming units (pfu), preferably by using the techniques described in U.S. Pat. No. 5,994,106.

### EXAMPLE 2

This example describes the generation of an adenovirus vector containing an expression cassette encoding a SEAP protein with a fused RGD sequence.

The SEAP coding sequence isolated from TROPIX™ pBC12/PL/SEAP VECTOR™, as described in Example 1, was further modified as follows. Two oligonucleotide sequences SEAPf.s (SEQ ID NO: 7) and SEAPf.a (SEQ ID NO: 8) were synthesized and annealed. These were filled in with dNTPs using Klenow polymerase and then blunt-end cloned into the HpaI site of pBC12/PL/SEAP. This resulted in a nucleic acid with 5′ and 3′ HindIII flanking sites and encoding a SEAP/RGD fusion protein (SEQ ID NOS: 9 and 10), wherein the portion encoding the RGD domain is flanked by Spe1 sites. Such Spe1 sites define a cassette that can be readily excised and replaced to generate additional SEAP fusion proteins. Additionally, the Spe1 sites can be employed to facilitate the insertion of additional sequences or linkers.

The HindIII SEAP sequence was cut out of the plasmid and cloned into the pAdCMV.MCS advenovirus transfer vector as described in Example 1, thereby creating plasmid pAD354CMV1-3.(SEAPfus) or pSEAPfus. This construct was then used to construct the adenovirus vector AdSEAPf, in a manner similar to the method set forth in Example 1.

### EXAMPLE 3

This example describes the generation of an adenovirus vector containing an expression cassette encoding a SEAP with a fused decorsin sequence.

As the decorsin protein is only 4.5 kD, the encoding sequence was fully synthesized and cloned into the pSEAPfus vector described in Example 2 to produce a cassette encoding a decorsin/SEAP fusion protein (SEQ ID NOS:11 and 12). The fusion gene from the pSAEPdecorsin plasmid was cut out with HindIII and then was cloned into the adenovirus transfer vector padCMV.MCS as described in Example 1 to create an adenovirus transfer plasmid pAD354CMV1-3.(SEAPdec). This plasmid was used to create the AdSEAPdec adenovirus vector using the methods described in Example 1.

### EXAMPLE 4

This example describes the generation of an adenovirus vector containing an expression cassette encoding a SEAP/HBNF fusion protein and the production of a vector containing such a polynucleotide.

Primers (SEQ ID NOS: 13 (the sense primer, having an SpeI site) and 14 (the antisense primer, having an XbaI site)) are used to generate a PCR product comprising a fragment of the HBNF gene (e.g., from plasmid pHHC12, which encodes residues 62-136 of human HBNF (Kretschmer et al., Growth Factors, 5, 99 (1991); Kretschmer et al., Biochem. Biophys. Res. Commun., 192(2), 420-29 (1993)), using the standard PCR technique. This fragment then is cut with SpeI/XbaI and cloned into the SpeI sites of pSEAPfus described in Example 2 (destroying one of the SpeI sites in the process). The resulting coding nucleic acid sequence and encoded fusion protein are set forth at SEQ ID NOS: 15 and 16. Adenoviral vectors comprising this construct (AdSEAP/HBNF) then can be produced by methods described herein and otherwise known to those of ordinary skill in the art.

### EXAMPLE 5

This example describes the generation of a polynucleotide encoding a SEAP/MK fusion protein and the production of a vector containing such a polynucleotide.

Primers (SEQ ID NOS: 17 (the sense primer, having an SpeI site) and 18 (the antisense primer, having an XbaI site)) are used to generate a PCR product comprising a fragment of the MK gene (e.g., from plasmid pMKHC4 (as described in Kretschmer et al., 1991 and 1993, supra)), which encodes human MK residues 59-123, using standard PCR techniques. This fragment then is cut with SpeI/XbaI and cloned into the SpeI sites of pSEAPfus as described in Example 4. The resulting coding nucleic acid sequence and encoded fusion protein are set forth at SEQ ID NOS: 19 and 20. Adenoviral vectors comprising this construct (AdSEAP/MK) then can be produced by methods described herein and otherwise known to those of ordinary skill in the art.

### EXAMPLE 6

This example describes the generation of a polynucleotide encoding a SEAP/VEGF₁₂₁ fusion protein and the production of a vector containing such a polynucleotide.

Primers (SEQ ID NOS: 21 (the sense primer, having an SpeI site) and 22 (the antisense primer, having an XbaI site)) are used to generate a PCR product comprising a fragment of the GF₁₂₁ gene (e.g., one of the pMT-VEGF plasmids described in U.S. Pat. No. 5,219,739), using standard PCR techniques. This fragment then is cut with SpeI/XbaI and cloned into the SpeI sites of pSEAPfus as described in Example 4 to produce pSEAP/VEGF₁₂₁. The resulting coding nucleic acid sequence and encoded fusion protein are set forth at SEQ ID NOS: 23 and 24. Adenoviral vectors comprising this construct (SEAP/VEGF₁₂₁) then can be produced by methods described herein and otherwise known to those of ordinary skill in the art.

### EXAMPLE 7

This example describes the generation of a polynucleotide encoding a SEAP/VEGF₁₂₁ fusion protein with a linker between the two protein domains, as well as the production of a vector containing such a polynucleotide.

Two oligonucleotides (SEQ ID NOS: 25 and 26) are synthesized and annealed (see Whitlow et al., Protein Eng, 6(8), 989-95 (1993)). The double-stranded oligonucleotide then is cloned into the SpeI site of pSEAP/VEGF₁₂₁, which maintains the SpeI site downstream wile destroying the up-stream SpeI site. The resulting construct pSEAP/W/VEGF₂₁ (SEQ ID NO: 27) encodes a fusion protein having both SEAP and VEGF₁₂₁ domains separated by a spacer (SEQ ID NO:28). Moreover, the remaining SpeI site can be employed for insertion of additional sequences and/or linkers. Adenoviral vectors comprising this construct (SEAP/W/VEGF₁₂₁) then can be produced by methods described herein and otherwise known to those of ordinary skill in the art.

### Incorporation by Reference

All references, including publications, patent applications, and patents, cited herein are hereby incorporated by reference to the same extent as if each reference were individually and specifically indicated to be incorporated by reference and were set forth in its entirety herein.

### Interpretation Guidelines

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for enhancing bone density or formation, the method comprising administering to at least one first cell associated with a region of a bone a first nucleic acid encoding a secreted alkaline phosphatase (SEAP), such that the first nucleic acid is expressed in the cell to produce the SEAP, whereby bone density or formation is enhanced within the region.

2. The method of claim 1, wherein at least one first nucleic acid is exposed to a cell in vivo in the region of the bone.

3. The method of claim 1, wherein at least one first nucleic acid is exposed to a cell ex vivo, which is then delivered in vivo to the region of the bone.

4. The method of claim 1, further comprising administering to a second cell associated with the region a second nucleic acid encoding an angiogenic protein, such that the second nucleic acid is expressed in the cell to produce the angiogenic protein.

5. The method of claim 4, wherein the angiogenic protein is a vascular endothelial growth factor, a connective tissue growth factor, an angiopoetin, an angiopoetin homologous protein, an angiogenin, an angiogenin-2, or P1 GF.

6. The method of claim 4, wherein the SEAP and the angiogenic protein are a single fusion protein comprising a first SEAP domain and a second angiogenic domain.

7. The method of claim 4, wherein the first cell and the second cell are the same.

8. The method of claim 4, wherein the first nucleic acid and the second nucleic acid are the same.

9. The method of claim 1, further comprising administering to a third cell associated with the region a third nucleic acid encoding an osteogenic protein, such that the third nucleic acid is expressed in the cell to produce the osteogenic protein.

10. The method of claim 9, wherein the osteogenic protein is selected from the group consisting of a bone morphogenic protein, a transforming growth factor, a latent transforming growth factor binding protein, latent membrane protein-1, a heparin-binding neurotrophic factor, growth and differentiation factor-5, a parathyroid hormone, a fibroblast growth factor, an epidermal growth factor, a platelet-derived growth factor, an insulin-like growth factor, a growth factor receptor, a cytokine, a chemotactic factor, a granulocyte/macrophage colony stimulating factor, a LIM mineralization protein, a leukemia inhibitory factor, a hedgehog protein, and midkine.

11. The method of claim 9, wherein the SEAP and the osteogenic protein are a single fusion protein comprising a first SEAP domain and a second osteogenic domain.

12. The method of claim 9, wherein the first cell and the third cell are the same.

13. The method of claim 9, wherein the first nucleic acid and the third nucleic acid are the same.

14. A viral vector comprising at least one first nucleic acid encoding a SEAP.

15. The viral vector of claim 14, which is an adenoviral vector.

16. The viral vector of claim 15, which is deficient in at least one gene function required for viral replication.

17. A bone graft comprising a first cell having a first exogenous nucleic acid encoding a SEAP.

18. The bone graft of claim 17, which further comprises a second cell having second exogenous nucleic acid encoding an angiogenic protein.

19. The bone graft of claim 18, wherein the SEAP and the angiogenic protein are a single fusion protein comprising a first SEAP domain and a second angiogenic domain.

20. The bone graft of claim 18, wherein the angiogenic protein is a vascular endothelial growth factor, a connective tissue growth factor, VEGF2, VEGF-C, a fibroblast growth factor, an angiopoetin, an angiopoetin homologous proteins, an angiogenin, an angiogenin-2, or PI GF.

21. The bone graft of claim 18, wherein the first cell and the second cell are the same.

22. The bone graft of claim 18, wherein the first nucleic acid and the second nucleic acid are the same.

23. The bone graft of claim 17, which further comprises a third cell having a third exogenous nucleic acid encoding an osteogenic protein.

24. The bone graft of claim 23, wherein the SEAP and the osteogenic protein are a single fusion protein comprising a first SEAP domain and a second osteogenic domain.

25. The bone graft of claim 23, wherein the osteogenic protein is selected from the group consisting of a bone morphogenic protein, a transforming growth factor, a latent transforming growth factor binding protein, latent membrane protein-1, a heparin-binding neurotrophic factor, growth and differentiation factor-5, a parathyroid hormone, a fibroblast growth factor, an epidermal growth factor, a platelet-derived growth factor, an insulin-like growth factor, a growth factor receptor, a cytokine, a chemotactic factor, a granulocyte/macrophage colony stimulating factor, a LIM mineralization protein, a leukemia inhibitory factor, a hedgehog protein, and midkine.

26. The bone graft of claim 23, wherein the first cell and the third cell are the same.

27. The bone graft of claim 23, wherein the first nucleic acid and the third nucleic acid are the same.

28. The bone graft of claim 17, which is an allograft or an autograft.

29. A recombinant expression cassette encoding a fusion protein having a first SEAP domain and a second angiogenic or osteogenic domain.

30. The recombinant expression cassette of claim 27, which is a SEAP/MK fusion protein, a SEAP/HBNF fusion protein, or SEAP/VEGF fusion protein.


SEQUENCE LISTING (28 sequences)

SEQ ID NO: 1 - DNA, 2747 bp, Homo sapiens
  CDS (9)..(1661)
catactccatgcccagaattcctgcctcgccactgtcctgctgccctccagacatgctggggccctgcatgctgctgctgctgctgctgctgggcctgaggctacagctctccctgggcatcatcctagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtgggggccgaaagtacatgtttcgcatgggaaccccagaccctgagtacccagatgactacagccaaggtgggaccaggctggacgggaagaatctggtgcaggaatggctggcgaagcgccagggtgcccggtacgtgtggaaccgcactgagctcatgcaggcttccctggacccgtctgtggcccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcggtccgtggtccccgcgttgcttcctctgctggccgggaccctgctgctgctggagacggccactgctccctgagtgtcccgtccctggggctcctgcttccccatcccggagttctcctgctccccgcctcctgtcgtcctgcctggcctccagcccgagtcgtcatccccggagtccctatacagaggtcctgccatggaaccttcccctccccgtgcgctctggggactgagcccatgacaccaaacctgccccttggctgctctcggactccctaccccaaccccagggactgcaggttgtgccctgtggctgcctgcaccccaggaaaggagggggctcaggccatccagccaccacctacagcccagtgggtaccaggcaggctcccttcctggggaaaagaagcacccagaccccgcgccccgctgatctttgcttcagtccttgaatcacctgtgggacttgaggactcgggatcttcaggacgcctggagaagggtggtttcctgccaccctgctggccaaggaggctcctggggtggggatcaccagggggattttgacacagccttcggctgccccccactaagttaattccacacccctgtaccccccagggggccctctgcctcatggcaaaggcttgccccaaatctcaacttctcagacgttccatacccccacatgccaatttcagcacccaactgagatccgaggagctcctgggaagccctgggtgcaggacactggtcgagagccaaaggtccctccccagacatctggacactgggcatagatttctcaagaaggaagactcccctgcctccccagggcctctgctctcctgggagacaaagcaataataaaaggaagtgtttgtaatcccagcactttgggaggccgaggtgggcggatcacgaggtcaggagatggagaccatcctggctaacacggtgaaaccccttatctatgcgcctgtagtcccagctacccaggaggctgaagcaggataatcgcttgaacccgggcggcggagattgcagtgagccgaggtcatgccactgcactgcagcctgggcgacagagcgagattctgcctcaaaaataaacaaataaattttaaaaataaataaataataaaaggaagtgttagac

SEQ ID NO: 2 - protein, 550 aa, Homo sapiens
MPRIPASPLSCCPPDMLGPCMLLLLLLLGLRLQLSLGIILVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVAHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 3 - DNA, 1581 bp, Homo sapiens
  CDS (1)..(1578)
atgcccagaattcctgcctcgccactgtcctgctgccctccagacatgctggggccctgcatgctgctgctgctgctgctgctgggcctgaggctacagctctccctgggcatcatcctagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtgggggccgaaagtacatgtttcgcatgggaaccccagaccctgagtacccagatgactacagccaaggtgggaccaggctggacgggaagaatctggtgcaggaatggctggcgaagcgccagggtgcccggtacgtgtggaaccgcactgagctcatgcaggcttccctggacccgtctgtggcccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggtga

SEQ ID NO: 4 - protein, 526 aa, Homo sapiens
MPRIPASPLSCCPPDMLGPCMLLLLLLLGLRLQLSLGIILVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVAHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPG

SEQ ID NO: 5 - DNA, 1918 bp, Homo sapiens
  CDS (11)..(1531)
aagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtccccgcgttgcttcctctgctggccgggaccctgctgctgctggagacggccactgctccctgagtgtcccgtccctggggctcctgcttccccatcccggagttctcctgctccccacctcctgtcgtcctgcctggcctccagcccgagtcgtcatccccggagtccctatacagaggtcctgccatggaaccttcccctccccgtgcgctctggggactgagcccatgacaccaaacctgccccttggctgctctcggactccctaccccaaccccagggactgcaggttgtgccctgtggctgcctgcaccccaggaaaggagggggctcaggccatccagccaccacctacagcccagtggcctcgagggatcc

SEQ ID NO: 6 - protein, 506 aa, Homo sapiens
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPG

SEQ ID NO: 7 - DNA, 41 bp, Artificial Sequence
  SEAPf.s primer oligonucleotide
cgactagttgctcttttggccgcggcgacattcgcaactgc

SEQ ID NO: 8 - DNA, 42 bp, Artificial Sequence
  SEAPf.a primer oligonucleotide
aagcttactagtttaacccgggtgcgcgcagttgcgaatgtc

SEQ ID NO: 9 - DNA, 1597 bp, Artificial Sequence
  SEAP/RGD fusion protein
aagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttcgactagttgctcttttggccgcggcgacattcgcaactgcgcgcacccgggttaaactagtaagctt

SEQ ID NO: 10 - protein, 524 aa, Artificial Sequence
  SEAP/RGD fusion protein
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGSTSCSFGRGDIRNCAHPG

SEQ ID NO: 11 - DNA, 1675 bp, Artificial Sequence
  SEAP/Decorsin fusion
aagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttcgactagtggatccgccccccgccttccgcagtgccagggcgacgaccaggaaaagtgcctttgcaacaaggacgaatgcccccccggccagtgccgctttccccgcggcgacgccgacccctactgcgaataatctagtaagctt

SEQ ID NO: 12 - protein, 550 aa, Artificial Sequence
  SEAP/Decorsin fusion
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGSTSGSAPRLPQCQGDDQEKCLCNKDECPPGQCRFPRGDADPYCE

SEQ ID NO: 13 - DNA, 30 bp, Artificial Sequence
  H2s SPE
gcgactagtcaatttggcgcggagtgcaaa

SEQ ID NO: 14 - DNA, 28 bp, Artificial Sequence
  H2a SPE
gcgtctagattaatccagcatcttctcc

SEQ ID NO: 15 - DNA, 1777 bp, Artificial Sequence
  SEAP HBNF fusion
aagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttcgactagtcaatttggcgcggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaagcagaatctaagaagaagaaaaaggaaggcaagaaacaggagaagatgctggattaatctagtaagctt

SEQ ID NO: 16 - protein, 584 aa, Artificial Sequence
  SEAP HBNF fusion
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGSTSQFGAECKYQFQAWGECDLNTALKTRTGSLKRALHNAECQKTVTISKPCGKLTKPKPQAESKKKKKEGKKQEKMLD

SEQ ID NO: 17 - DNA, 28 bp, Artificial Sequence
  M2s SPE
gcgactagtgagtttggagccgactgca

SEQ ID NO: 18 - DNA, 28 bp, Artificial Sequence
  M2a SPE
gcgtctagacctagtcctttcccttccc

SEQ ID NO: 19 - DNA, 1748 bp, Artificial Sequence
  SEAP/MK fusion
aagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttcgactagtgagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactaggtctagtaagctt

SEQ ID NO: 20 - protein, 574 aa, Artificial Sequence
  SEAP/MK fusion
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGSTSEFGADCKYKFENWGACDGGTGTKVRQGTLKKARYNAQCQETIRVTKPCTPKTKAKAKAKKGKGKD

SEQ ID NO: 21 - DNA, 30 bp, Artificial Sequence
  V/s SPE
gcgactagtgcacccatggcagaaggagga

SEQ ID NO: 22 - DNA, 30 bp, Artificial Sequence
  V/a SPE
gcgtctagatcaccgcctcggcttgtcaca

SEQ ID NO: 23 - DNA, 1915 bp, Artificial Sequence
  SEAP/VEGF121 fusion
aagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttcgactagtgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtgatctagtaagctt

SEQ ID NO: 24 - protein, 630 aa, Artificial Sequence
  SEAP/VEGF121 fusion
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGSTSAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 25 - DNA, 60 bp, Artificial Sequence
  Ws SPE
ctagaggcagtacttcgggcagtggtaagcctggtagtggtgagggtagtactaagggta

SEQ ID NO: 26 - DNA, 60 bp, Artificial Sequence
  Wa SPE
ctagtacccttagtactaccctcaccactaccaggcttaccactgcccgaagtactgcct

SEQ ID NO: 27 - DNA, 1975 bp, Artificial Sequence
  SEAP/W/VEGF121 fusion
aagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttcgactagaggcagtacttcgggcagtggtaagcctggtagtggtgagggtagtactaagggtactagtgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtgatctagtaagctt

SEQ ID NO: 28 - protein, 650 aa, Artificial Sequence
  SEAP/W/VEGF121 fusion
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGSTRGSTSGSGKPGSGEGSTKGTSAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR